(19) United States
(12) Patent Application Publication
(10) Pub. No.: US 2006/0034920 A1
(43) Pub. Date: Feb. 16, 2006

(54) Effervescent formulations of oxytocin

(76) Inventor: Finn Larsen, Edinburgh (GB)

Correspondence Address: Edwin V Merkel;Nixon Peabody, Clinton Square, P O Box 31051, Rochester, NY, 14603-1051, US

(21) Appl. No.: 10/527,879

(22) PCT Filed: Sep. 19, 2003
(86) PCT No.: PCT/GB03/04146

§ 371 Date: Aug. 10, 2005

(30) Foreign Application Priority Data
Sep. 19, 2002 (GB) 0221712.3

(51) Int. Cl. A61K 38/22; A61K 9/46

(52) U.S. Cl. 424/466; 514/9

(57) ABSTRACT

An effervescent formulation comprising oxytocin, preferably comprising multilayer effervescent microspheres containing an acidic substance, a basic substance and water-soluble isolating agent. An effervescent formulation comprising oxytocin wherein dissolution in water of the multilayer effervescent microspheres leads, after almost immediate effervescence, to a solution or a homogeneous dispersion of the oxytocin. The formulation is used for the induction or augmentation of labour or treatment or prevention of postpartum haemorrhage or treatment of missed abortion or facilitation of lactation.

This invention relates to formulations of oxytocin and their use in the induction or augmentation of labour during parturition.

The induction of labour was applied in 18% of all U.S. deliveries in recent years (Stubbs, 2000, Clin Obstet Gynecol 43, 489-94). One reason for the application of this technique is that perinatal mortality increases in pregnancies over 42 weeks, but can be reduced by the induction of labour (Alfirevic Z and Walkinshaw S A, 1994, Br J Hosp Med 52,218-21).

Most commonly, oxytocin and prostaglandins and synthetic prostaglandin are used to induce labour. Intravenous prostaglandin E2 and F2 alpha can be used to induce labour by ripening of the cervix. However, their use is associated with higher rates of maternal side effects and uterine hyperstimulation than oxytocin (Luckas M, Bricker L, 2000, Cochrane Database Syst Rev 4, CD002864).

Oxytocin is a neurohypophyseal peptide hormone that induces labour and lactation in mammals (Yuan et al, 2002 J Med Chem 45, 2512-2519). Oxytocin is the commonest labour induction agent used worldwide (Kelly and Tan, 2001, Cochrane Database Syst Rev, 3, CD003246).

Oxytocin (OT) binds to specific receptors of myometrial cells, inducing and increasing myometrial contractions. During pregnancy and especially close to term, an increase in the myometrial OT-receptor concentration is found, leading to an increased sensitivity of the myometrium towards circulating OT. The factors determining the receptor level are not completely understood, but may include the level of steroids, OT- and oestrogen receptors. Because of the increased sensitivity, only a small increase in the maternal OT blood-level is necessary to induce myometrial contractions at term. The level of maternal plasma OT does not change significantly throughout pregnancy. The fetus is found to secrete considerable amounts of OT during the first stage of labour, which reaches the myometrium in spite of the high level of oxytocinase in placenta. At the second stage of labour the distension of the lower birth canal might cause release of OT from the maternal neurohypophysis into the blood, increasing the myometrial contractions. This mechanism is observed in animals, but not established in the human (Giraldi et al, 1990, Dan Med Bull, 37, 377-83).

For the induction and augmentation of labour, oxytocin is presently administered by slow intravenous infusion preferably by means of an infusion pump. Once labour is progressing, oxytocin infusion may be gradually withdrawn.

Oxytocin has also been given, as the citrate, in the form of a buccal tablet to induce labour; however, absorption is irregular following buccal administration and this route has been superseded by intravenous administration.

A first aspect of the invention provides an effervescent formulation comprising oxytocin. It may be used for the induction or augmentation of labour.

By ‘effervescent formulation’ we mean that the formulation is effervescent when placed in an aqueous solution.

By ‘oxytocin’ we include a cyclic nonapeptide having the structure of the hormone produced by the posterior lobe of the pituitary that stimulates the contraction of the uterus: it has the structure Cys-Tyr-Ile-Gln-Asn-Cys-Pro-Leu-Gly-NH2 cyclic (116) disulphide; [2-leucine, 7-isoleucine]vasopressin; has a CAS registry number 50-56-6 and is also called alpha-hypophamine and oxytocinum. Also included are salts thereof, including acetate and citrate.

Effervescent formulations offer an advantage over the existing forms of supplying oxytocin as they have a high level of patient acceptability. Effervescent formulations also give a more consistent pharmacokinetic profile than buccal delivery. The formulations may be placed on the tongue where they effervesce, and release the oxytocin.

A preferred embodiment of the invention is that the effervescent formulation comprises multilayer effervescent microspheres. The manufacture of certain suitable multilayer effervescent microspheres is described in WO 98/31342 and U.S. Pat. No. 6,210,711 B1, hereby incorporated by reference in their entirety.

A still further embodiment of the invention is that the multilayer effervescent microspheres contain an acidic substance, a basic substance and water-soluble isolating agent.

The term ‘microsphere’ will be intended to refer to microgranules formed of a support material consisting of a matrix in which the oxytocin is dispersed. In accordance with the European Pharmacopoeia monograph on spheres, microspheres have an average diameter of less than 1.0 mm and greater than or equal to 1.0 μm. They are generally intended for oral or parenteral administration and are used either as constituents of pharmaceutical form, such as tablets, or in their natural form combined or otherwise with other excipients, and distributed or otherwise in unit doses, such as sachets, gel-capsules or powder for injectable preparation.

The ‘water-soluble isolating agent’ may be any such agent which serves as both a binder and as an isolating barrier intended to avoid an effervescent reaction between the alkaline substance and the acidic substance during the preparation process but also during storage of the microspheres, irrespective of the storage conditions. Typically, it is chosen from polyvinylpyrrolidone, hydroxypropyl cellulose, methyl cellulose, lactose and sucrose.

By ‘acidic substance’ we include a powder of acidic nature containing an organic acid, for example citric acid, ascorbic acid or acetylleucine.

By ‘basic substance’ we mean a powder of alkaline nature containing a sodium bicarbonate or any other carbonate usually used in the preparation of effervescent forms, such as lithium hydrogen carbonate, monosodium carbonate, lithium glycine carbonate, monopotassium carbonate, calcium carbonate or magnesium carbonate. It is preferred that the ‘basic substance’ is a sodium salt such as sodium bicarbonate.

A preferred embodiment of the invention relates to multilayer effervescent microspheres containing an acidic substance, a basic substance and a water-soluble isolating agent whose dissolution in water leads, after almost immediate effervescence, to a solution or a homogeneous dispersion of oxytocin.

According to a first variant of this embodiment of the invention, the water-soluble isolating agent is dispersed in the entire bulk of each microsphere, the latter having a two-layer structure: a layer of acidic substance in which is dispersed the water-soluble isolating agent and a layer of alkaline substance in which is dispersed the water-soluble isolating agent.

According to a second variant of this embodiment of the invention, the water-soluble isolating agent is in the form of a thin film separating the acidic and alkaline substances. In this case, each microsphere has a three-layer structure: a layer of acidic substance and a layer of alkaline substance separated by a layer of water-soluble isolating agent.

Whether the microspheres have a two-layer or three-layer structure, the water-soluble isolating agent serves two purposes; it acts as a binder and as an isolating barrier intended to avoid an effervescence reaction between the alkaline substance and the acidic substance during the preparation process but also during storage of the microspheres, irrespective of the storage conditions.

In a preferred embodiment of the invention the effervescent formulation contains oxytocin present in a unit dose amount of from about 50 ng to 100 μg such as 50 ng, 100 ng, 200 ng, 340 ng, 500 ng, 750 ng, 1 μg, 2 μg, 3 μg, 4 μg, 5 μg, 6 μg, 7 μg, 8 μg, 9 μg, 10 μg, 11 μg, 15 μg, 20 μg, 30 μg, 50 μg, 75 μg or 100 μg. Most preferably the oxytocin is present in a unit dosage amount of about 340 ng to about 11 μg.

In a further embodiment the effervescent formulation of the invention is presented in a tablet form. Methods of forming tablets suitable for the invention from such an effervescent formulation are well known to those skilled in the art. Methods of forming tablets suitable for the invention from such an effervescent formulation are well known to those skilled in the art. A tablet may be made by compression or moulding, optionally with one or more accessory ingredients. Compressed tablets may be prepared by compressing in a suitable machine the active ingredient in a free-flowing form such as a powder or granules. Moulded tablets may be made by moulding in a suitable machine a mixture of the powdered compound moistened with an inert liquid diluent.

In a further embodiment the effervescent formulation of the invention is presented in a powder form. Methods of forming powders suitable for the invention from such an effervescent formulation are well known to those skilled in the art.

It is preferred that when the formulation contains microspheres, the oxytocin is not present within the microspheres. For example, when microspheres are tabletted to form a tablet, the oxytocin is preferably present on or between the microspheres in the tablet.

The oxytocin may, however, in some embodiments, be present in the microspheres.

A further aspect of the invention is a process for making an effervescent formulation containing oxytocin.

A preferred embodiment of the invention is a process wherein the effervescent formulation comprises multilayer effervescent microspheres containing an acidic substance, a basic substance, and a water-soluble isolating agent which upon dissolution in water leads, after almost immediate effervescence, to a solution or a homogeneous dispersion of oxytocin.

In a preferred embodiment of the invention, the oxytocin is not present within the microspheres. In a further embodiment of the process of the invention the acidic and/or basic substances contains or contain oxytocin.

In a further preferred embodiment of the process of the invention the process employs the method of rotary granulation in a fluidized air bed.

The advantage of rotary granulation applied to these effervescent compositions is the continuous linking of the operations in one and the same chamber which, as a result of the components used and certain precautions taken, induces no effervescence. Furthermore, this rotary granulation technique allows the relative proportions of the various compounds to be modified, in particular the relative molar proportions of the acidic and basic fractions.

Specifically, the process according to the invention makes it possible advantageously to obtain effervescent forms whose relative proportion of alkaline and acidic fractions is less than the stoichiometric proportion implemented in the prior art for effervescent tablets manufactured by the granulation method, without the quality of the effervescence being adversely affected.

In particular, the relative proportion of the basic and acidic substances implemented in the context of the process according to the invention is less than 0.6, in particular less than 0.25.

All the steps of the process according to the invention are carried out under atmospheric pressure, without any specific dehydration system or any specific precautions.

The apparatus used to carry out the process for preparing the effervescent microspheres is, for example, apparatus constructed by the company Glatt, onto which a rotor tank is fitted.

Such an item of apparatus is described in patent EP 0,505,319, which we include, by way of reference, in the present application.

Also subject of the present invention is, firstly, a process for preparing effervescent microspheres which have a two-layer structure according to the first variant described above.

Said process is performed by rotary granulation in a fluidized air bed combined with a system for spraying powder and a system for the tangential spraying of wetting liquid. The process comprises two continuous steps, a first step of spheronization of microspheres using a powder A and a second step of spheronization of a powder B on the microspheres of powder A, one of the powders A and B being acidic and the other alkaline and it being possible for each of them to contain or consist of oxytocin. It is preferred that powders A or B contain but do not consist of oxytocin.

During the first spheronization, the powder A is placed in the moving rotary granulation tank and suspended in the air bed. The components of the powder A are mixed together for five minutes and the air inlet temperature is stabilised to a temperature T₀.

The powder A thus blended is sprayed with a wetting liquid containing the water-soluble isolating agent. The microspheres of powder A obtained are dried by bringing the air inlet temperature to Ts and are then optionally screened using a 1000 μm screen. During the second spheronization, the air inlet temperature is brought to T₀. The powder B and the wetting liquid containing the water-soluble isolating agent are then simultaneously sprayed onto the dried powder A microspheres obtained previously. The powder B is sprayed by means of the powder spraying system installed on the Glatt apparatus. The two-layer microspheres obtained are dried by bringing the air inlet temperature to Ts. After drying, the microspheres must be packaged quickly, but a small amount of moisture uptake does not harm the storage.

During the two spheronizations, the wetting liquid containing the water-soluble isolating agent is the same, for example polyvinylpyrrolidone (PVP) dissolved in an alcohol or an aqueous-alcoholic mixture, in particular PVP dissolved to 4% by weight in ethanol at 60% by volume.

The two-layer microspheres obtained according to the process of the invention have an average particle size of between 20 and 500 μm.

A subject of the present invention is also a process for preparing effervescent microspheres which have a three-layer structure according to the second variant described above.

Said process is performed according to the method of rotary granulation in a fluidized air bed combined with a system for the tangential spraying of wetting liquid.

The process comprises three continuous steps, a first step of spheronization of microspheres using a powder A, a second step of spheronization of a water-soluble isolating agent on the microspheres of powder A, and then a third step of spheronization of a powder B on the microspheres A protected with a film of water-soluble isolating agent, one of the powders A and B being acidic and the other alkaline and it being possible for each of them to contain or consist of oxytocin. It is preferred that powders A or B contain but do not consist of oxytocin.

During the first spheronization, the powder A containing an added binder, for example PVP, is placed in the moving tank and suspended in the air bed. The components of the powder A are mixed together for five minutes and the air inlet temperature is stabilized to T₀. The powder A thus blended is sprayed with a wetting liquid. The microspheres of powder A obtained are dried by bringing the air inlet temperature to Ts. During the second spheronization, the air inlet temperature is brought to T₀. The water-soluble isolating agent is added directly to the tank and the wetting liquid sprayed until microspheres of powder A which are coated with a film of water-soluble isolating agent are obtained, and are dried by bringing the air inlet temperature to Ts. After drying, the coated microspheres are screened and the powder B is then added directly to the rotary granulation tank when the air inlet temperature has stabilized at T₀. The three-layer microspheres are obtained by spraying the preceding microspheres with a wetting liquid. The three-layer microspheres obtained are dried by bringing the air inlet temperature to Ts. After drying, the microspheres must be packaged quickly, but a small amount of moisture uptake does not harm the storage.

During the first two steps, the wetting liquid is, for example, an aqueous-alcoholic solution, in particular ethanol at 60% by volume. During the final step, the water-soluble isolating agent can be introduced by means of the powder B, in which case the wetting liquid used will be the same as during the first two steps, or alternatively the isolating agent is introduced by means of the wetting liquid, which will be an alcoholic or aqueous-alcoholic solution containing the isolating agent, for example PVP dissolved to 4% by weight in ethanol at 60% by volume.

The three-layer microspheres obtained according to the process of the invention have an average particle size of between 200 and 1000 μm.

According to the process for manufacturing microspheres, whether they are two-layer or three-layer microspheres, the powder of alkaline nature contains a sodium bicarbonate or any other carbonate usually used in the preparation of effervescent forms, such as lithium hydrogen carbonate, monosodium carbonate, lithium glycine carbonate, monopotassium carbonate, calcium carbonate, magnesium carbonate and, optionally oxytocin; whereas the powder of acidic nature contains an organic acid, for example citric acid, ascorbic acid, acetylleucine and, optionally, oxytocin. It is preferred that the oxytocin is not present within the microspheres, but rather is present on or between them in the final formulation (typically a tablet). In some embodiments, however, the powder of alkaline nature and the powder of acidic nature contain but do not consist of oxytocin.

In a further embodiment of the process of the invention the acidic and alkaline powders can also contain a diluent, for example lactose or Glucidex; flavorings and sweeteners, for example orange flavoring, citric acid, sodium saccharinate; various excipients.

In a preferred embodiment of the process of the invention oxytocin is present such that the resulting effervescent formulation contains oxytocin present in a unit dose amount of from between 50 ng to 100 μg such as 50 ng, 100 ng, 200 ng, 340 ng, 500 ng, 750 ng, 1 μg, 2 μg, 3 μg, 4 μg, 5 μg, 6 μg, 7 μg, 8 μg, 9 μg, 10 μg, 11 μg, 15 μg, 20 μg, 30 μg, 50 μg, 75 μg or 100 μg. Most preferably the oxytocin is present in a unit dosage amount of 340 ng to 11 μg.

In a further embodiment of the process of the invention the effervescent formulation of the invention is presented in a tablet form. Methods of forming tablets suitable for the invention from such an effervescent formulation are well known to those skilled in the art as described above.

Preferably, the oxytocin is present in the tablet on or between microspheres (when present).

According to one embodiment of the invention, the powder A is of alkaline nature and the powder B is of acidic nature.

According to another embodiment of the invention, the powder B is of alkaline nature and the powder A of acidic nature.

The wetting liquid is sprayed by means of a nozzle 1.2 mm in diameter, at an average flow rate of between 10 and 30 g/min. The air inlet temperature of the fluidized bed is between 55 and 65° C. during the spheronization steps (T₀) and between 75 and 85° C. during the drying phases (Ts).

The microspheres obtained according to the process of the invention contain 5 to 75% of alkaline substance, 10 to 75% of acidic substance, 3 to 15% of water-soluble isolating agent, 5 to 50% of diluent and 1 to 30% of flavorings and sweeteners and contain an appropriate amount of oxytocin such as 0.00003% to 0.02%.

The relative humidity of the microspheres obtained according to the process of the invention, measured for fifteen minutes by the infrared balance method at 90° C., is between 1 and 2% at the rotary granulation tank outlet.

The overall yield for the process is calculated from the fraction of particles smaller than 2500 μm in size, the working yield of the spheres corresponds to the fraction of particles between 200 and 1000 μm, for the process for preparing three-layer microspheres, between 20 and 500 μm for the process for preparing two-layer microspheres.

The feasibility of the process according to the invention is evaluated according to the ease with which the microspheres are obtained, the speed of production of a batch and the yield for each step.

Analysis of the batches includes particle size analysis of a sample of 100 g of spheres by the superimposed screens method (sample obtained from the total fraction of a batch), after which a morphological study of the microspheres obtained, relating to the overall appearance, sphericity, cohesion and uniformity of the particles, is carried out by examination with a binocular magnifying glass.

According to one variant of the invention, the two-layer or three-layer effervescent microspheres are manufactured by the mounting technique combined with a system for the tangential spraying of wetting liquid. The powder A and the powder B can be mounted successively on spheres containing oxytocin coated with water-soluble isolating agent, or on neutral spheres.

A further aspect of the invention is an effervescent formulation of oxytocin obtained or obtainable by any one of the processes of the invention mentioned above.

A further aspect of the invention provides an effervescent formulation of oxytocin for use in medicine.

A further aspect of the invention provides a pharmaceutical composition which comprises an effervescent formulation of oxytocin according to the invention and a pharmaceutically acceptable carrier.

A further aspect of the invention is a method of induction or augmentation of labour, or treatment or prevention of postpartum haemorrhage or treatment of missed abortion or facilitation of lactation comprising administering an effervescent formulation of oxytocin according to the invention and/or obtained or obtainable by a process according to the invention.

A further aspect of the invention is the use of an effervescent formulation of oxytocin according to the invention and/or obtained or obtainable by a process according to the invention in the manufacture of a medicament for the induction or augmentation of labour or treatment or prevention of postpartum haemorrhage or treatment of missed abortion or facilitation of lactation.

Preferred embodiments of the invention are described in the following processes.

Process 1: Process for preparing multilayer effervescent microspheres containing an acidic substance, a basic substance, and a water-soluble isolating agent which upon dissolution in water leads, after almost immediate effervescence, to a solution or a homogeneous dispersion of oxytocin, wherein the acidic and basic substances contain or consist of oxytocin, which employs the method of rotary granulation in a fluidized air bed.

Process 2. Process for preparing microspheres defined in process 1, which employs the method of rotary granulation in a fluidized air bed combined with a system for spraying powder and a system for the tangential spraying of wetting liquid, which comprises two continuous steps, a first step of spheronization of microspheres using a powder A and a second step of spheronization of a powder B on the microspheres of powder A, one of the powders A and B being acidic and the other alkaline.

Process 3. Process according to process 2, wherein the powder A is introduced directly into the rotary granulation tank and then sprayed with a wetting liquid containing the water-soluble isolating agent, while the powder B and a wetting liquid containing the water-soluble isolating agent are simultaneously and respectively sprayed via the system for spraying powder and the system for the tangential spraying of liquid.

Process 4. Process according to process 3, wherein the microspheres obtained have an average particle size of between 20 and 500 μm.

Process 5. Process for preparing microspheres as defined in process 1, which employs the method of rotary granulation in a fluidized air bed combined with a system for the tangential spraying of wetting liquid, which comprises three continuous steps, a first step of spheronization of microspheres using a powder A, a second step of spheronization of a water-soluble isolating agent on the microspheres of powder A, and then a third step of spheronization of a powder B on the microspheres A protected with a film of water-soluble isolating agent, one of the powders A and B being acidic and the other alkaline.

Process 6. Process according to process 5, wherein the powder A and the water-soluble isolating agent are sprayed with an alcoholic or aqueous-alcoholic solution.

Process 7. Process according to process 5, wherein the powder B contains the water-soluble isolating agent and is sprayed with an alcoholic or aqueous-alcoholic solution.

Process 8. Process according to process 5, wherein the powder B is sprayed with a wetting liquid containing the water-soluble isolating agent.

Process 9. Process according to process 5, wherein the microspheres obtained have an average particle size of between 200 and 1000 μm.

Process 10. Process according to process 3, wherein the wetting liquid containing the water-soluble isolating agent is polyvinylpyrrolidone dissolved in an alcohol or an aqueous-alcoholic mixture, which is polyvinylpyrrolidone dissolved to 4% by weight in ethanol at 60% by volume.

Process 11. Process according to process 2 or 5, wherein the powder of alkaline nature contains a sodium bicarbonate or another carbonate used in the preparation of effervescent forms, selected from lithium hydrogen carbonate, monosodium carbonate, lithium glycine carbonate, monopotassium carbonate, calcium carbonate, magnesium carbonate; and oxytocin.

Process 12. Process according to process 2 or 5, wherein the powder of acidic nature contains citric acid or ascorbic acid or, acetylleucine, and/or oxytocin.

Process 13. Process according to process 1, wherein the powder of alkaline nature also contain an edible diluent and; flavorings and sweeteners.

Process 14. Process according to process 2 or 5, wherein the microspheres obtained contain 5 to 75% of alkaline substance, 10 to 75% of acidic substance, 3 to 15% of water-soluble isolating agent, 5 to 50% of diluent, and 1 to 30% of flavorings and sweeteners.

Process 15. Process according to process 2 or 5, wherein the powder A is of alkaline nature and the powder B of acidic nature.

Process 16. Process according to process 2 or 5, wherein the powder A is of acidic nature and the powder B of alkaline nature.

Process 17. Process according to process 3 or 6, wherein the wetting liquid is sprayed by means of a nozzle 1.2 mm in diameter, at an average flow rate of between 10 and 30 g/min.

Process 18. Process according to process 2 or 5, wherein the air inlet temperature of the fluidized bed is between 55 and 65° C. during spheronization steps, and between 75 and 85° C. during drying phases associated with the spheronization steps.

Process 19. Process according to process 2 or 5, wherein the relative humidity of the microspheres obtained is between 1 and 2% at the rotary granulation tank outlet.

Process 20. Process for preparing microspheres as defined in process 1, which employs the mounting technique combined with a system for the tangential spraying of wetting liquid.

Process 21. Process according to process 20, wherein the powder A and the powder B are mounted successively on spheres containing oxytocin coated with water-soluble isolating agent, or on neutral spheres.

Process 22. Process according to process 12, wherein the powder of acidic nature also contains an edible diluent and flavorings and sweeteners.

The examples which follow illustrate the invention without limiting its scope.

The percentages are expressed on a weight basis.

### EXAMPLE 1

### Two-Layer Effervescent Microspheres Containing Ascorbic Acid (Vitamin C)

Alkaline microspheres are prepared, on which is deposited the acidic substance (vitamin C).

The table below gives the details of the formulation used.

| FORMULATION | COMPONENT | PERCENTAGE |
|---|---|---|
| Powder A | | |
| Alkaline compound | Sodium | 20% |
| | bicarbonate | |
| Diluent | Lactose | 6% |
| Sweetener | Glucidex 6 .RTM. | 6% |
| Powder B | | |
| Acidic compound | Ascorbic acid | 49.99994% |
| Oxytocin | | 0.00006% |
| Flavoring | Orange flavoring | 1% |
| Sweeteners | Sodium | 0.3% |
| | saccharinate | |
| | Glucidex 6 .RTM. | 6.35% |
| Diluent | Lactose | 6.35% |

The wetting liquid used during the two successive rotary granulations is an aqueous-alcoholic PVP solution containing 4% PVP in ethanol at 60% by volume.

This mixture is sprayed at an average flow rate of 25 grams per minute.

In this formulation, the lactose is combined in equal part with Glucidex 60, although it is possible to use lactose alone.

The powder formulations A and B are prepared on batches of variable size of 1000 to 5000 g with, depending on the case, use of equipment from the company Glatt.

The effervescent spheres obtained have a fairly uniform appearance and a majority particle size of fractions between 200 and 500 μm. The relative humidity is 1.6% at the rotary granulation tank outlet.

### EXAMPLE 2

### Two-Layer Effervescent Microspheres Containing Acetylleucine

Alkaline microspheres are prepared, on which is deposited the acidic substance (acetylleucine) under the same conditions as in Example 1.

The table below gives the details of the formulation used.

| FORMULATION | COMPONENT | PERCENTAGE |
|---|---|---|
| Powder A | | |
| Alkaline compound | Sodium | 20% |
| | bicarbonate | |
| Diluent | Lactose | 9.85% |
| Powder B | | |
| Acidic compound | Acetylleucine | 49.98% |
| Oxytocin | | 0.02% |
| Flavoring | Orange flavoring | 1% |
| Sweetener | Sodium | 0.3% |
| | saccharinate | |
| Diluent | Lactose | 9.85% |

The particle size distribution of the batch is a majority for the fractions 25 to 500 μm.

The relative humidity is 1.9% at the rotary granulation tank outlet. According to the size of the batches ranging from 1000 to 10,000 g, apparatus GPCG 1 or GPCG 5 from the company Glatt with a rotor tank mounting [lacuna].

### EXAMPLE 3

### Three-Layer Effervescent Microspheres Containing Ascorbic Acid (Vitamin C)

Three-layer effervescent microspheres are manufactured, comprising an alkaline core isolated from the acidic substance, ascorbic acid, by means of a film of PVP.

| FORMULATION | COMPONENT | PERCENTAGE |
|---|---|---|
| Powder A | | |
| Alkaline compound | Sodium | 25% |
| | bicarbonate | |
| Binder | PVP K30 | 1.316% |
| Diluent | Lactose | 7.950% |
| Water-soluble | PVP K30 | 6.958% |
| isolating agent | | |
| Powder B | | |
| Acidic compound | Ascorbic acid | 49.998% |
| Oxytocin | | 0.002% |
| Flavoring | Orange flavoring | 1% |
| Sweeteners | Sodium | 0.2% |
| | saccharinate | |
| | Citric acid | 1% |
| Diluent | Lactose | 6.950% |

The test is carried out in apparatus of GPCG1 type from the company Glatt, with the rotor tank mounting.

1460 g of ethanol at 60% by volume are sprayed in total during the three steps, at an average flow rate of 15 grams per minute.

The size of the final batch is 1000 g.

The working yield corresponding to the fraction of particles between 200 and 1000 μm is 65%. The relative humidity is 1.5% at the tank outlet.

### EXAMPLE 4

### Preparation of Effervescent Tablets Containing Oxytocin

Effervescent tablets containing between 1 μg to 10 μg oxytocin are prepared so that the time for drug dissolution or tablet disintegration and/or dissolution is less than 10 minutes.

In the laboratory scale manufacturing process, oxytocin is mixed with effervescent microspheres prepared as described above with a Glatt GPCG1. The mixture is then added with diluent (mannitol), lubricants (magnesium stearate, talc), flavouring and tabletted on a single punch alternative press under isolator.

In the industrial scale process, oxytocin is introduced directly on the effervescent microspheres directly in the Glatt by the powder device. After drying, the spheres are mixed with the other excipients and tabletted.

### EXAMPLE 5

### Induction or Augmentation of Labour with Oxytocin

A female patient presenting symptoms of delayed labour is treated with an effervescent formulation according to Example 1 which has been made into a tablet.

The patient is supplied with an effervescent formulation containing 340 ng of oxytocin in the form of a 500 mg tablet. The quantity of oxytocin used is dependent on the severity of the condition and the tolerance of the patient to oxytocin.

The patient places the tablet on the tongue. The tablet effervesces and delivers the oxytocin to the patient.

The oxytocin may be supplied to the patient in this manner every 30 minutes (with or without an escalating dose) until labour is progressing. In any event the skilled practitioner will be able to ascertain the amount and frequency of the doses to produce the required effect.

### EXAMPLE 6

### Induction or Augmentation of Labour with Oxytocin

A female patient presenting symptoms of delayed labour is treated with an effervescent formulation according to Example 2 which has been made into a tablet.

The patient is supplied with an effervescent formulation containing 11 μg of oxytocin in the form of a 50 mg tablet. The quantity of oxytocin used is dependent on the severity of the condition and the tolerance of the patient to oxytocin.

The patient places the tablet on the tongue. The tablet effervesces and delivers the oxytocin to the patient.

The oxytocin may be supplied to the patient in this manner every 30 minutes (with or without an escalating dose) until labour is progressing. In any event the skilled practitioner will be able to ascertain the amount and frequency of the doses to produce the required effect.

1. An effervescent formulation comprising oxytocin.

2. An effervescent formulation according to claim 1 comprising multilayer effervescent microspheres.

3. An effervescent formulation according to claim 2 wherein the multilayer effervescent microspheres contain an acidic substance, a basic substance and water-soluble isolating agent.

4. An effervescent formulation according to claim 3 wherein dissolution in water of the multilayer effervescent microspheres leads, after almost immediate effervescence, to a solution or a homogeneous dispersion of the oxytocin.

5. An effervescent formulation according to claim 4 wherein the water-soluble isolating agent is dispersed in the entire bulk of each microsphere, the latter having a two-layer structure: a layer of acidic substance in which is dispersed the water-soluble isolating agent and a layer of alkaline substance in which is dispersed the water-soluble isolating agent.

6. An effervescent formulation according to claim 4 wherein the water-soluble isolating agent is in the form of a thin film separating the acidic and alkaline substances such that each microsphere has a three-layer structure: a layer of acidic substance and a layer of alkaline substance separated by a layer of water-soluble isolating agent.

7. An effervescent formulation according to claim 1 wherein the oxytocin is present in a unit dose amount of from 50 ng to 100 μg.

8. An effervescent formulation according to claim 7 wherein the oxytocin is present in a unit dose amount of 340 ng to 11 μg.

9. An effervescent formulation according to claim 1 wherein the formulation is presented in a tablet form.

10. An effervescent formulation according to claim 1 wherein the formulation is presented in a powder form.

11. An effervescent formulation according to claim 1 wherein the oxytocin is present within a microsphere.

12. An effervescent formulation according to claim 1 wherein the oxytocin is not present within a microsphere.

13-14. (canceled)

15. A pharmaceutical composition comprising an effervescent formulation according to claim 1 and a pharmaceutically acceptable carrier.

16. A process for making an effervescent formulation containing oxytocin.

17. A process according to claim 16 wherein the effervescent formulation comprises multilayer effervescent microspheres containing an acidic substance, a basic substance, and a water-soluble isolating agent which upon dissolution in water leads, after almost immediate effervescence, to a solution or a homogeneous dispersion of oxytocin.

18. A process according to claim 17 wherein the acidic and/or basic substances contains or contain oxytocin.

19. A process according to claim 16 further comprising:
forming microspheres, wherein the oxytocin is not present in the microspheres.

20. A process according to claim 18 which employs the method of rotary granulation in a fluidized air bed.

21. A process according to claim 17 wherein basic substance also contains an edible dilutant and/or flavourings and/or sweeteners.

22. A process according to claim 17 wherein the oxytocin is present in an amount to give from 50 ng to 100 μg in the final unit dosage form.

23. A process according to claim 22 wherein the oxytocin is present in an amount to give from 340 ng to 11 μg in the final unit dosage form.

24. A process according to claim 16 further comprising preparing the microspheres into a tablet.

25. A process according to claim 24 wherein the oxytocin is present on or between the microspheres in the tablet.

26. An effervescent formulation of oxytocin obtained or obtainable by the process of claim 16.

27. A method of induction or augmentation of labour or treating or preventing postpartum haemorrhage or treating missed abortion or facilitation of lactation comprising administering an effervescent formulation of oxytocin according to claim 1.

28. (canceled)

29. The method according to claim 27, wherein the effervescent formulation is present in a pharmaceutical composition that includes a pharmaceutically acceptable carrier.
